SEQUENCE LISTING (8 sequences)

SEQ ID NO: 1 - protein, 230 aa, Batis maritima
MSTVANIAPVFTGDCKTIPTPEECATFLYKVVNSGGWEKCWVEEVIPWDLGVPTPLVLHLVKNNALPNGKGLVPGCGGGYDVVAMANPERFMVGLDISENALKKARETFSTMPNSSCFSFVKEDVFTWRPEQPFDFIFDYVFFCAIDPKMRPAWGKAMYELLKPDGELITLMYPITNHEGGPPFSVSESEYEKVLVPLGFKQLSLEDYSDLAVEPRKGKEKLARWKKMNN

SEQ ID NO: 2 - protein, 246 aa, Batis maritima
MASAIVDVAGGGRQQALDGSNPAVARLRQLIGGGQESSDGWSRCWEEGVTPWDLGQPTPAVVELVHSGTLPAGDATTVLVPGCGAGYDVVALSGPGRFVVGLDICDTAIQKAKQLSAAAAAAADGGDGSSSFFAFVADDFFTWEPPEPFHLIFDYTFFCALHPSMRPAWAKRMADLLRPDGELITLMYLAEGQEAGPPFNTTVLDYKEVLNPLGLVITSIEDNEVAVEPRKGMEKIARWKRMTKSD

SEQ ID NO: 3 - protein, 203 aa, Batis maritima
MAGPTTEFWQERFEKKETGWDRGSPSPQLLAWLASGALRPCRIAVPGCGSGWEVAELAQRGFDVVGLDYTAAATTRTRALCDARGLKAEVLQADVLSYQPEKKFAAIYEQTCLCAIHPDHWIDYARQLHQWLEPQGSLWVLFMQMIRPAATEEGLIQGPPYHCDINAMRALFPQKDWVWPKPPYARVSHPNLSHELALQLVRR

SEQ ID NO: 4 - protein, 201 aa, Batis maritima
MENVNQAQFWQQRYEQDSIGWDMGQVSPPLKAYIDQLPEAAKNQAVLVPGAGNAYEVGYLHEQGFTNVTLVDFAPAPIAAFAERYPNFPAKHLICADFFELSPEQYQFDWVLEQTFFCAINPSRRDEYVQQMASLVKPNGKLIGLLFDKDFGRDEPPFGGTKDEYQQRFATHFDIDIMEPSYNSHPARQGSELFIEMHVKD

SEQ ID NO: 5 - protein, 226 aa, Batis maritima
MAEVQQNSGNSNGENIIPPEDVAKFLPKTVDEGGWEKCWEDGVTPWDQGRATPLVVHLVESSSLPLGRGLVPGCGGGHDVVAMASPERYVVGLDISESALEKAAETYGSSPKAKYFTFVKEDFFTWRPNELFDLIFDYVVFCAIEPETRPAWAKAMYELLKPDGELITLMYPITDHDGGPPYKVAVSTYEDVLVPVGFKAVSIEENPYSIATRKGKEKLARWKKIN

SEQ ID NO: 6 - protein, 193 aa, Batis maritima
MNLSADAWDERYTNNDIAWDLGEVSSPLKAYFDQLENKEIKILIPGGGNSHEAAYLFENGFKNIWVVDLSETAIGNIQKRIPEFPPSQLIQGDFFNMDDVFDLIIEQTFFCAINPNLRADYTTKMHHLLKSKGKLVGVLFNVPLNTNKPPFGGDKSEYLEYFKPFFIIKKMEACYNSFGNRKGRELFVILRSK

SEQ ID NO: 7 - protein, 207 aa, Batis maritima
MSDPTQPAVPDFETRDPNSPAFWDERFERRFTPWDQAGVPAAFQSFAARHSGAAVLIPGCGSAYEAVWLAGQGNPVRAIDFSPAAVAAAHEQLGAQHAQLVEQADFFTYEPPFTPAWIYERAFLCALPLARRADYAHRMADLLPGGALLAGFFFLGATPKGPPFGIERAELDALLTPYFDLIEDEAVHDSIAVFAGRERWLTWRRRA

SEQ ID NO: 8 - protein, 276 aa, Batis maritima
MTDQSTLTAAQQSVHNTLAKYPGEKYVDGWAEIWNANPSPPWDKGAPNPALEDTLMQRRGTIGNALATDAEGNRYRKKALVPGCGRGVDVLLLASFGYDAYGLEYSGAAVQACRQEEKESTTSAKYPVRDEEGDFFKDDWLEELGLGLNCFDLIYDYTFFCALSPSMRPDWALRHTQLLAPSPHGNLICLEYPRHKDPSLPGPPFGLSSEAYMEHLSHPGEQVSYDAQGRCRGDPLREPSDRGLERVAYWQPARTHEVGKDANGEVQDRVSIWRRR

(19) United States
(12) Patent Application Publication
(10) Pub. No.: US 2016/0194683 A1
(43) Pub. Date: Jul. 7, 2016

(54) Gas Biosensors

(71) Applicant: William Marsh Rice University, Houston, TX (US)

(72) Inventors: Jonathan SILBERG, Houston, TX (US); Caroline A. MASIELLO, Houston, TX (US); Hsiao-Ying CHENG, Houston, TX (US)

(21) Appl. No.: 15/075,433

(22) Filed: Mar. 21, 2016

Related U.S. Application Data
(62) Division of application No. 14/512,379, filed on Oct. 10, 2014, now abandoned.
(60) Provisional application No. 61/890,736, filed on Oct. 14, 2013.

(51) Int. Cl. C12Q 1/48

(52) U.S. Cl. CPC C12Q 1/48

(57) ABSTRACT

Microbial biosensors that generate gas outputs in hard-to-image materials using exogenous methyl halide transferase (WIT) genes. By varying the promoter that is fused to the MHT gene, biosensors for different triggers can be made.

### PRIOR RELATED APPLICATIONS

This application is a divisional of U.S. application Ser. No. 14/512,379, filed Oct. 10, 2014, which claims priority to U.S. Ser. No. 61/890,736, filed Oct. 14, 2013. Both are incorporated herein by reference in its entirety for all purposes.

### FEDERALLY SPONSORED RESEARCH STATEMENT

Not applicable.

### FIELD OF THE DISCLOSURE

The disclosure generally relates to novel biosensors, based on detection of a gas that can diffuse out of the cell and the matrix containing the cell (e.g., soil, sediment, biofilm, muds, blood, oil and the like).

### BACKGROUND OF THE DISCLOSURE

Many microbes can be genetically modified to detect a property within their environment and report this detection by producing an easy to measure molecular output, like a fluorescent molecule (e.g. GFP). While our ability to build microbial biosensors has exploded with the growth of synthetic biology, these biosensors have not yet seen widespread use in the earth science community, including soil science, marine science, and the petroleum industry, mainly due to complications associated with the petri-dish-to-porous media transition. Specifically, existing microbial biosensor outputs, like fluorescence, are challenging to detect in solid matrices, such as soils, sediments, biofilms and the like.

Thus, what is needed in the art are robust biosensors that function outside the constraints of a petri plate or flask environment, and can be used in real samples in real field situations. Preferably, reporting methods, devices and systems that are not light based and that can be detected without disrupting the matrix in which the cell resides should be developed.

### SUMMARY OF THE DISCLOSURE

The disclosure describes a novel reporter system using a gas-reporting biosensor. A gas producing genetic circuit can be inserted into diverse environmental microbes to program them to release very low levels of an easily detected gas (e.g., a volatile organic molecule) in response to some environmental or cellular trigger. The gas can then be monitored using approaches similar to those routinely used to monitor CO₂ in the plant-soil environment, e.g., gas chromatography, and this can be done without disturbing the matrix or other environment in which the cell resides.

FIG. 1 illustrates the reaction catalyzed by a methyl halide transferase (MHT), which we have selected for our gas reporter gene constructs. MHTs use halide ions and a ubiquitous microbial metabolite, S-adenosyl methionine, to synthesize volatile methyl halides (FIG. 1). The volatile methyl halides (CH₃X) are well-suited as outputs for microbial biosensors because the methyl halide gas can diffuse out the cell, and out of the cell's environmental matrix, and then be detected by conventional gas detection methods. MHTs are not prevalent within bacteria, but they can be expressed as functional enzymes in bacteria (12). In fact, a recent study showed that over eighty different MHTs can be used to program Escherichia coli to produce high levels of methyl halides in this non-native host species (13).

FIG. 2 illustrates a prototype biosensor shown herein to be functional. This biosensor achieves gas reporting by using a methyl halide transferase (rather than GFP) as an output for signal-dependent promoters. An exemplary assay in soil is shown, but the same basic methodology can be applied to any hard-to-image cell media or matrix.

This new class of volatile gas reporter outputs will have advantages over existing reporters in studying any hard-to-image materials, such as soil biogeochemical processes, because a volatile reporter can be detected without disrupting soil structure and could provide dynamic data on changes in the levels of molecules that microbes are programmed to sense.

The biosensors described herein directly report microbial behavior in response to environmental stimuli. They also provide data dynamically and nondestructively, and these data will be from the perspective of the microbes. These benefits are significant compared to traditional sensors. For example, while it is possible to determine environmental concentrations of microbially reactive materials like O₂, NO₃⁻, NH₄⁺, PO₄³⁻, metals like Hg, and reactive minerals with traditional tools, these measurements do not necessarily provide information about whether microbes are receiving sufficient quantities of these reactants to drive changes in microbial-plant-soil interactions.

The gas-reporting biosensors developed herein are transferrable to other microbes and other laboratories, allowing the production of new microbial tools to help address problems ranging from detecting hydrocarbon contamination, toxic metals, and nutrient availability to reporting on the activation of cellular pathways involved in specific biological processes. We anticipate these biosensors will be useful to diverse communities whose industries are influenced by microbial behavior in hard-to-image materials, e.g., the soil science, oceanographic, petroleum communities and any other applications where the microbes are hard to image because of their local environment.

The reporters described herein have a wide variety of applications, including measuring:

Transcriptional activity within a cell: measuring promoter activities (transcription) in any hard-to-image matrixes (e.g., soils, sediments, biochars, complex and/or opaque feedstocks, etc.).

Level of chemical compounds in the environment: measuring chemical levels in soils, sediments, or other matrixes being remediated, e.g., superfund sites and oil spills, and evaluating the benefits of different remediation processes.

Presence of microbes in the environment: rapid and sensitive detection of microbes in food to avoid foodborne illness, as well as detection of changes in the levels of beneficial (symbionts) and deleterious (pathogens) upon amendment of soils with various materials (biochar, fertilizers, pesticides, etc.), also waste water treatment.

Metabolic status of biochemical reactions: optimizing microbial strains for converting complex feedstocks like beet molasses into fine chemicals and biofuels, also using activated sludge in wastewater as a complex feedstock.

Microbial signaling and decision-making within a population: understanding outcomes of various soil manipulations that have beneficial and deleterious effects to make profitable land use decisions.

Microbial sensing within hard-to-image matrixes: reporting on cellular reactions in biological systems within complex materials where the gas produced can escape both the cell, and the matrix housing the cells.

Among the MHTs tested herein, a variety of methyl halide products were observed, with some enzymes being generalists capable of producing different methyl halides (CH₃Cl, CH₃Br, and CH₃I) with similar efficiencies, and other enzymes being specialists, only synthesizing one methyl halide (CH₃Cl, CH₃Br, or CH₃I) with high efficiency (13). Taken together with the paucity of MHTs discovered through bacterial genome sequencing projects and the easy measurement of CH₃X by gas chromatography, we predict that members of this enzyme family will be useful as volatile gas reporters in microbial biosensors created using diverse soil organisms (gram negative and positive bacteria, fungi and plants).

The gas biosensors described here are made by fusing various natural promoters (that are on/off or dose-dependent in their response to what the cell senses in an environment) to a methyl halide transferase gene, and transforming soil or other organisms (bacteria, fungi, etc.) with these vectors so that the organism produces methyl halides when the promoter is switched on by an environmental signal as shown in FIG. 1. The MHT gene can be maintained as a plasmid or other vector, but are preferably integrated into the genome using an integration vector suitable for the host species. In that way, no selection system will be needed, making the biosensors more useful in material environments. We have already shown that promoter MHT fusion can be chromosomally incorporated into microbes, facilitating stable transformation of soil organisms into biosensors.

Work to date indicates that the reporter gene can be used in E. coli and yeast, and given the diversity of yeast and E. coli, it is predicted that the method will be useful in most bacteria, especially those lacking endogenous MHTs, or whose native MHT genes are inactivated or reduced in activity. Indeed, similar biosensors have already been developed in diverse species including yeast, E. coli, Pseudomonas putida, Burkholderia sartisoli, Erwinia herbicola, Bacillus subtilis, Lactobacillus lactis and Salmonella Typhimurium, to name a few (see Table 2). Any of these biosensors can be easily converted by merely swapping out the existing reporter gene with an MHT gene.

We anticipate that it may be necessary to generate a range of reporting gases, as some will be more appropriate for specific environments.

Our preliminary reporting gases (CH₃Br, CH₃Cl, and CH₃I) can have toxic effect at high enough concentrations, although at the levels tested herein the reporters were functional. If needed, toxicity can be avoided by simply tuning the maximal output down to a level that is non-toxic for the organisms in the system being studied.

A variety of different reporter gene constructs have been built, and are described below. Exemplary MHT that can be used as reporters herein include the methyl halide transferases below and their homologs.

| Organism | Kingdom | Acc. No. |
|---|---|---|
| Batis maritima | Plantae | AAD26120 or AAK73255 |
| Oryza sativa | Plantae | EAY92545 or AAS07345.1 |
| Rhodoferax ferrireducens | Bacteria | YP_522685 |
| Psychrobacter cryohalolentis | Bacteria | YP_581342 |
| Brassica oleracea | Plantae | AAK69761 |
| Polaribacter irgensii | Bacteria | ZP_01117536 |
| Burkholderia Xenovorans | Bacteria | YP_557005 |
| Arabidopsis thaliana | Plantae | AF109128_1 |
| Aspergillus niger | Plantae | CAK43983 |

Other species known to have MHT genes include: Burkholderia phymatum STM815; Synechococcus elongatus PCC 63011; Brassica rapa subsp. chinensis; Brassica oleracea TM1; Brassica oleracea TM2; Arabidopsis thaliana TM1 and TM2; Leptospirillum zsp. Group II UBA; Cryptococcus neoformans var. JEC21; Ostreococcus tauri; Dechloromonas aromatica RCB; Coprinopsis cinerea okayama; Robiginitalea bofirmata HTCC2501; Maricaulis maris MCS10; Flavobacteria bacterium BBFL7; Vitis vinifera and Halorhodospira halophila SL1, to name a few. The genes can be easily located by search of GenBank or UniProt or other dates and Bayer (2009) provides additional examples.

Suitable MHTs are not limited to proteins encoded by naturally occurring genes. For example, techniques of directed evolution can be used to produce new or hybrid gene products with methyl transferase activity. In addition, catalytically active fragments and variants of naturally occurring MHTs can be used. Partially or wholly synthetic MHTs, such as enzymes designed in silico or produced by using art-known techniques for directed evolution including gene shuffling, family shuffling, staggered extension process (StEP), random chimeragenesis on transient templates (RACHITT), iterative truncation for the creation of hybrid enzymes (ITCHY), recombined extension on truncated templates (RETT), and the like (see Crameri et al., 1998, “DNA shuffling of a family of genes from diverse species accelerates directed evolution” Nature 391:288-91; Rubin-Pitel et al., 2006, “Recent advances in biocatalysis by directed enzyme evolution” Comb Chem High Throughput Screen 9:247-57; Johannes and Zhao, 2006, “Directed evolution of enzymes and biosynthetic pathways” Curr Opin Microbiol. 9:261-7; Bornscheuer and Pohl, 2001, “Improved biocatalysts by directed evolution and rational protein design” Curr Opin Chem. Biol. 5:137-43), Pandey N. et al., Combining random gene fission and rational gene fusion to discover near-infrared fluorescent protein fragments that report on protein-protein interactions, ACS Synth. Biol., Just Accepted Manuscript (2014), each incorporated by reference it its entirety for all purposes.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention, such as instructions for use, buffers, and the like.

The term “quorum sensing” refers to the measurement of the population density of a particular species.

The term “regulated promoter” as used herein is defined to exclude constitutive promoters. “Constitutive promoters,” in contrast, are always in an “on” state, expressing the open reading frames under their control at >80% capacity.

The phrase “growth in a solid matrix” refers to cells growing in soils, sediments, sands, biochar, biofilm, and the like, which have a significant solid content (at least 50% or 75% solid content based on dry weight) and which are housing the organisms of interest. The term specifically excludes cells grown on the surface of e.g., a petri plate, and requires some degree of intermingling of the cells within the solid matrix.

By partially-opaque media, what is meant is that the media is insufficiently clear to allow the more traditional light bases sensors, such GFP, RFP and luciferase, to be used (e.g., allowing >50% or >75% of visible light through a 1 cm sample). Examples of partially opaque media include soil, sediment, most vertebrate organisms, mixed biofuel feedstocks, crude petroleum, blood, mud, and the like.

The following abbreviations are used herein:

| ABBREVIATION | TERM |
|---|---|
| AHL | Acyl homoserine lactone-activates the |
| | lasR promoter |
| CAT | chloramphenicol acetyltransferase |
| Cherry | An RFP |
| GFP | Green fluorescent protein |
| lasL | Gene encoding LasL- |
| lasR | The gene encoding LasR-a transcription |
| | activator. |
| MBT | Methyl bromide transferase |
| MCT | Methyl chloride transferase |
| MHT | Methyl halide transferase |
| LasI | A Pseudomonas aeruginosa protein that |
| | produces AHL |
| LasR | A Pseudomonas aeruginosa transcription |
| | activator that actives Pₗₐₛ while bound |
| | to its ligand AHL |
| P | promoter |
| Pₗₐₛ | A promoter sequence recognized by LasR |
| RBS | Ribosomal binding site |
| RFP | Red fluorescent protein |

The invention includes any one or more of the flowing embodiments, in any combination:

A method, comprising: growing a microorganism comprising an exogenous gene encoding an MHT in a solid matrix or a partially opaque medium, wherein said gene is under the direct or indirect control of a promoter-of-interest; adding a substrate for said MHT to said solid matrix or a partially opaque medium; capturing a gas released by said microorganism; and measuring an amount of said captured gas, said amount being proportional to an activity of said promoter-of-interest.

A method, comprising: growing a microorganism comprising an genomically integrated exogenous gene encoding an MHT in a solid matrix or a partially opaque medium; adding a substrate for said MHT to said solid matrix or a partially opaque medium; capturing a gas released by said microorganism; and measuring an amount of said captured gas, said amount being proportional to an activity of said promoter-of-interest.

A method of detecting the activity of a promoter-of-interest, comprising adding a microorganism comprising an exogenous gene encoding an MHT to a sample, wherein said MHT gene is under the direct or indirect control by a promoter-of-interest, adding a halide salt to said sample, incubating said sample until said halide salt is converted to a gaseous methyl halide, and detecting an amount of said methyl halide gas being emitted from said sample, wherein said amount directly correlates to an activity level of said promoter-of-interest.

A microbial biosensor for a ligand, comprising a microorganism having a ligand activated promoter operatively coupled to a gene encoding a methyl halide transferase, such that when said ligand and a halide salt are present, said ligand binds said promoter, activating expression of said methyl halide transferase, which converts said halide salt to a gaseous methyl halide which can then be detected, thus detecting said ligand.

A quorum sensitive microbial biosensor, comprising a microbe having a constitutive promoter operably linked to a gene whose expression produces a ligand that can exit said microbe, said microbe also comprising a ligand activated promoter operatively coupled to a gene encoding a methyl halide transferase, such that when a quorum of microbes are present to make sufficient ligand, said ligand binds said promoter, activating expression of said methyl halide transferase, which converts a halide ion to a methyl halide which can then be detected, thus detecting said ligand and indicating a quorum of said microbes.

A stress sensitive microbial biosensor, comprising a microorganism having a stress sensitive promoter operably linked to a gene encoding a methyl halide transferase, such that when said microorganism is under stress, said stress sensitive promoter activates expression of said methyl halide transferase, which converts a halide ion to a gaseous methyl halide which can then be detected, thus detecting said stress.

A toxin microbial biosensor, comprising a microorganism having a constitutive promoter operably linked to a gene whose expression produces a toxin sensing protein that produces a ligand, said microorganism also comprising a ligand activated promoter operatively coupled to a gene encoding a methyl halide transferase, such that when enough toxin is present to make sufficient ligand, said ligand binds said promoter, activating expression of said methyl halide transferase, which converts a halide ion to a methyl halide which can then be detected, thus detecting said toxin.

A method or biosensor wherein said substrate is a chlorine or bromine or iodine ion.

A method or biosensor wherein said exogenous gene encoding said MHT is integrated into the genome.

A method or biosensor wherein a second exogenous gene is added to make a ligand for activating the promoter-of-interest.

A method or biosensor wherein said promoter-of-interest is a metal-sensing promoter.

A method or biosensor wherein said promoter-of-interest is a stress-sensing promoter.

A method or biosensor wherein said promoter-of-interest is a redox-sensing promoter.

A method or biosensor wherein said promoter-of-interest is a estrogen- or androgen-responsive promoter and wherein said microorganism also comprises an exogenous gene for an estrogen receptor or an androgen receptor.

A method or biosensor wherein said promoter-of-interest is an aromatic hydrocarbon responsive promoter.

A method or biosensor wherein said promoter-of-interest is a benzene, toluene, and xylene (BETX) responsive promoter and wherein said microorganism also comprises an exogenous gene for a transcriptional activator XylR.

A method or biosensor wherein said exogenous gene is from Batis maritima or the MHT has a sequence selected from:

SEQ ID NO: 1:
MSTVANIAPV FTGDCKTIPT PEECATFLYK VVNSGGWEKC
WVEEVIPWDL GVPTPLVLHL VKNNALPNGK GLVPGCGGGY
DVVAMANPER FMVGLDISEN ALKKARETFS TMPNSSCFSF
VKEDVFTWRP EQPFDFIFDY VFFCAIDPKM RPAWGKAMYE
LLKPDGELIT LMYPITNHEG GPPFSVSESE YEKVLVPLGF
KQLSLEDYSD LAVEPRKGKE KLARWKKMNN
SEQ ID NO: 2
MASAIVDVAG GGRQQALDGS NPAVARLRQL IGGGQESSDG
WSRCWEEGVT PWDLGQPTPA VVELVHSGTL PAGDATTVLV
PGCGAGYDVV ALSGPGRFVV GLDICDTAIQ KAKQLSAAAA
AAADGGDGSS SFFAFVADDF FTWEPPEPFH LIFDYTFFCA
LHPSMRPAWA KRMADLLRPD GELITLMYLA EGQEAGPPFN
TTVLDYKEVL NPLGLVITSI EDNEVAVEPR KGMEKIARWK
RMTKSD
SEQ ID NO: 3
MAGPTTEFWQ ERFEKKETGW DRGSPSPQLL AWLASGALRP
CRIAVPGCGS GWEVAELAQR GFDVVGLDYT AAATTRTRAL
CDARGLKAEV LQADVLSYQP EKKFAAIYEQ TCLCAIHPDH
WIDYARQLHQ WLEPQGSLWV LFMQMIRPAA TEEGLIQGPP
YHCDINAMRA LFPQKDWVWP KPPYARVSHP NLSHELALQL
VRR
SEQ ID NO: 4
MENVNQAQFW QQRYEQDSIG WDMGQVSPPL KAYIDQLPEA
AKNQAVLVPG AGNAYEVGYL HEQGFTNVTL VDFAPAPIAA
FAERYPNFPA KHLICADFFE LSPEQYQFDW VLEQTFFCAI
NPSRRDEYVQ QMASLVKPNG KLIGLLFDKD FGRDEPPFGG
TKDEYQQRFA THFDIDIMEP SYNSHPARQG SELFIEMHVK
D
SEQ ID NO: 5:
MAEVQQNSGN SNGENIIPPE DVAKFLPKTV DEGGWEKCWE
DGVTPWDQGR ATPLVVHLVE SSSLPLGRGL VPGCGGGHDV
VAMASPERYV VGLDISESAL EKAAETYGSS PKAKYFTFVK
EDFFTWRPNE LFDLIFDYVV FCAIEPETRP AWAKAMYELL
KPDGELITLM YPITDHDGGP PYKVAVSTYE DVLVPVGFKA
VSIEENPYSI ATRKGKEKLA RWKKIN
SEQ ID NO: 6
MNLSADAWDE RYTNNDIAWD LGEVSSPLKA YFDQLENKEI
KILIPGGGNS HEAAYLFENG FKNIWVVDLS ETAIGNIQKR
IPEFPPSQLI QGDFFNMDDV FDLIIEQTFF CAINPNLRAD
YTTKMHHLLK SKGKLVGVLF NVPLNTNKPP FGGDKSEYLE
YFKPFFIIKK MEACYNSFGN RKGRELFVIL RSK
SEQ ID NO: 7
MSDPTQPAVP DFETRDPNSP AFWDERFERR FTPWDQAGVP
AAFQSFAARH SGAAVLIPGC GSAYEAVWLA GQGNPVRAID
FSPAAVAAAH EQLGAQHAQL VEQADFFTYE PPFTPAWIYE
RAFLCALPLA RRADYAHRMA DLLPGGALLA GFFFLGATPK
GPPFGIERAE LDALLTPYFD LIEDEAVHDS IAVFAGRERW
LTWRRRA
and
SEQ ID NO: 8
MTDQSTLTAA QQSVHNTLAK YPGEKYVDGW AEIWNANPSP
PWDKGAPNPA LEDTLMQRRG TIGNALATDA EGNRYRKKAL
VPGCGRGVDV LLLASFGYDA YGLEYSGAAV QACRQEEKES
TTSAKYPVRD EEGDFFKDDW LEELGLGLNC FDLIYDYTFF
CALSPSMRPD WALRHTQLLA PSPHGNLICL EYPRHKDPSL
PGPPFGLSSE AYMEHLSHPG EQVSYDAQGR CRGDPLREPS
DRGLERVAYW QPARTHEVGK DANGEVQDRV SIWRRR.

### BRIEF DESCRIPTION OF THE DRAWINGS

FIG. 1. MHTs catalyze the production of methyl halides.

FIG. 2. Concept for microbial biosensors that generate gas outputs.

FIG. 3A shows the experimental design and vector construct for demonstrating proof of concept using a constitutive promoter.

FIG. 3B shows methyl bromide data obtained with three different integration strains of E. coli. The cells were transformed with an integration vector (pOSIP) containing the Batis maritima MHT gene under a constitutive promoter (P₁₄) and one three RBS's with different strengths (BCD2, BCD8, BCD14). MG1655=base strain of E. coli K12 (F-lambda-ilvG-rfb-50 rph-1); MG1655-19=MG1655 with integrated plas-MHT and pCat-LasR; MG1655-27-1=MG1655 with integrated p14_BCD2_MHT; MG1655-27-2=MG1655 with integrated p14_BCD8_MHT; MG1655-27-3=MG1655 with integrated p14_BCD14_MHT; MHT=methyl halide transferase from Batis maritima (Acc. No. Q9ZSZ7); p14=a constitutive promoter (promoters described in PMID: 23474465); CD2=a bicistronic design RBS; BCD8=a bicistronic design RBS; BCD14=a bicistronic design RBS; lasR gene=Acc. No.BAA06489 LasR [Pseudomonas aeruginosa]; Pₗₐₛ=LasR regulated promoter; P_{Cat}=a constitutive promoter (BBa_I14033) from biobrick registry.

FIG. 4A-C show that Biochars inhibit cell-cell communication to differing extents in pure culture, wherein 4A is an agar plate assay, 4B shows the spotting diagram of senders and receiver cells, and 4C is images of the agar plates. LasR=transcriptional activator of PlasR; PlasR=lasR promoter; GFP=green fluorescent protein; MHT=methyl halide transferase; Cherry=a red fluorescent protein; AHL=acyl homoserine lactone.

FIG. 5. Genetic circuit that programs bacteria to report on AHL by producing a methyl halide within hard-to-image materials, such as soils. LasR=transcriptional activator of PlasR; PlasR=lasR promoter; GFP=green fluorescent protein; MCT=methyl chloride transferase; MBT=methyl bromide transferase; AHL=acyl homoserine lactone; Pon=constitutive promoter.

FIG. 6. Examples of biological processes that are regulated by quorum sensing.

FIG. 7A shows the experimental design and vector constructs for a ligand activated MHT reporter. The ligand is AHL, which binds to the LasR made by the constitutive upstream promoter on the lasR gene.

FIG. 7B shows the results obtained with this vector, adding varying amounts of AHL to the cells using both Cl (middle panel) and Br (lower panel) as substrates for the MHT. The GFP control is in the upper panel. LasR=transcriptional activator of PlasR; PlasR=lasR promoter; GFP=green fluorescent protein; MHT=methyl halide transferase; AHL=acyl homoserine lactone; Pon=constitutive promoter.

FIG. 8A shows the experimental design of a cellular redox state biosensor. The production of reporter gases indicates the depletion of NADPH and a decreased NADPH/NADP⁺ ratio in cells.

FIGS. 8B and 8C are the experimental results proving that our experimental design can report on cellular redox states for E. coli EW11 (FIG. 8B) and E. coli CS50 (FIG. 8C). pSenNADPH=a plasmid encoding NADPH biosensor (pACYCDuet-1 backbone+pSoxRS intergenic region E. coli K12+Venus); pET28B=a T7-IPTG inducible expression plasmid vector from EMD Biosciences; pJTA03=a ferredoxin expression plasmid (pET28b+soFdx); pJTA04=a ferredoxin expression plasmid (pET28b+crFdxl); soFd=Plant type [2Fe-2S] Ferredoxin from Spinacia oleracea; crFd=Plant type [2Fe-2S] Ferredoxin from Chlamydomonas reinhardtii; SoxR=Superoxide Response protein (NADPH dependent iron sulfur containing transcription factor); RsxABCDGE=operon for the soxR reducing complex; RseC=SoxR iron-sulfur cluster reduction factor component; ecFNR=ferredoxin-NADPH reductase from E. coli; FNR=ferredoxin-NADPH reductase; (FNR+)=E. Coli CS50(DE3); (FNR−)=E. coli EW11 (Δfpr).

FIG. 9A shows the vectors and experimental design for a dual MHT and optional light based reporter system, wherein the light produced by the Cherry RFP can be used if desired to control for cell density.

FIG. 9B shows the CH₃Br/cell density ratio with time, wherein optical density at 600 nm (OD) was the control for cell growth. LasR=transcriptional activator of PlasR; PlasR=lasR promoter; Plac=lac promoter; MHT=methyl halide transferase; AHL=acyl homoserine lactone; Pon=constitutive promoter.

FIG. 10A shows the experimental setup for testing to ensure that the biosensor will work, even when in the presence of a solid matrix, such as soil or sand.

FIG. 10B shows the data obtained with the various matrices (soil, sand and clay).

FIG. 10C shows an exemplary AHL titration with cells housed in sand.

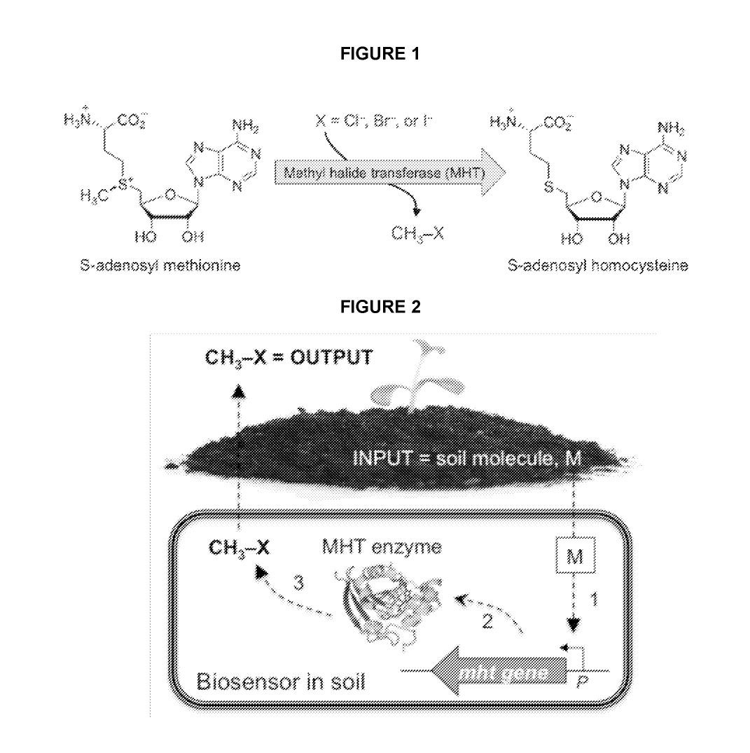

### DETAILED DESCRIPTION

A number of gas reporting biosensors have been built and tested herein. The following descriptions are exemplary only, and not intended to unduly limit the claims.

### Proof of Concept

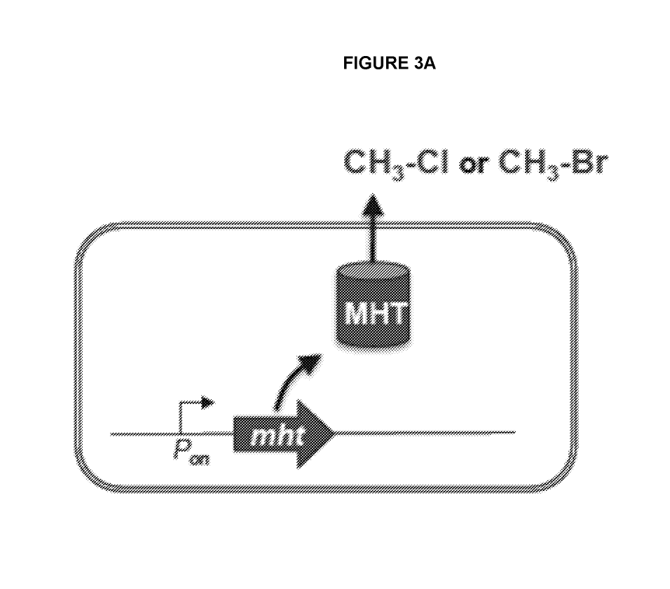

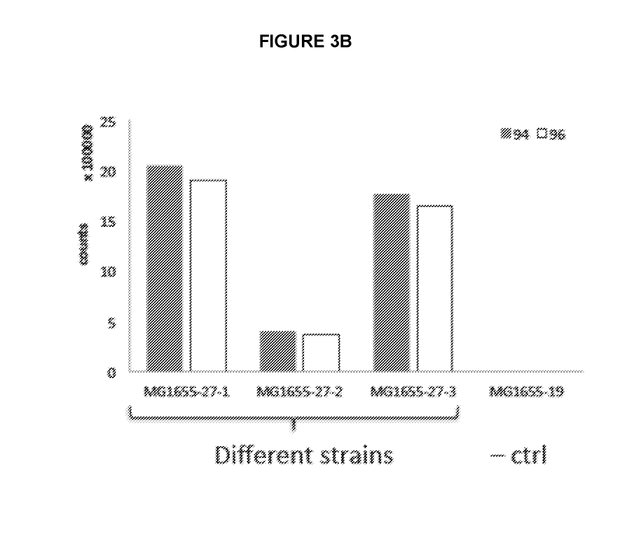

Constitutive MHT reporter gene constructs were prepared as shown in FIG. 3A for proof of concept studies. Seven MHT genes from Batis maritima, Oryza sativa; Rhodoferax ferrireducens, Psychrobacter cryohalolentis, Brassica oleracea, Polaribacter irgensii, and Burkholderia Xenovorans were synthesized and cloned into an expression vector under an inducible promoter. Transformed E. coli were grown in LB plus 200 mM NaCl or KBr and production of methyl halide gas measured in the headspace by GC-MS (data not shown). In these initial experiments, the B. maritima MHT showed the highest activity with either Cl⁻ or Br⁻ ions as substrate, and was chosen for continued work.

### Genomic Integration

Three strains carrying an integrated Batis maritima MHT gene (BmMHT) were created by coupling the BmMHT gene to a constitutive promoter (P₁₄) and three ribosomal binding sites (RBS) with different strengths (BCD2, BCD8, BCD14) in the integration vector pOSIP. Each was integrated into the phage 186 attachment site within the genome of E. coli MG1655 using the site specific recombinase from phage 186. Strains having the desired chromosomal insertions were verified by PCR.

Cells with a genomic copy of the MHT construct under control of the three different RBS were then tested for MHT activity by growing the cells in LB plus 200 mM KBr. Headspace gas was collected at 5 hrs, and CH₃Br measured by GC-MS. The results are shown in FIG. 3B.

Neither the bromine ions, nor the CH₃Br gas were toxic and all cells grew well. The CH₃Br gas was readily detectable in the headspace, whereas the uninduced vector control cells produced no gas at all.

This simple experiment demonstrated that the concept of using MHTs as reporter genes is feasible in E. coli. The strains harboring MHT under the control of a constitutive promoter all showed high constitutive production, albeit to varying levels depending on the RBC used. The ability of E. coli to tolerate constitutive production indicates that CH₃Br production is not lethal to cells at these high levels. Note: the control AHL inducible promoter (lasR promoter) showed no activity in this system, because it was not induced in this experiment.

### Yeast Expression

Methyl halide expression was also demonstrated in yeast S. cerevisae (US20110151534). Thus, there is at least proof of concept for use of the gas reporter in yeast.

### Integrated Ligand Activated MHT

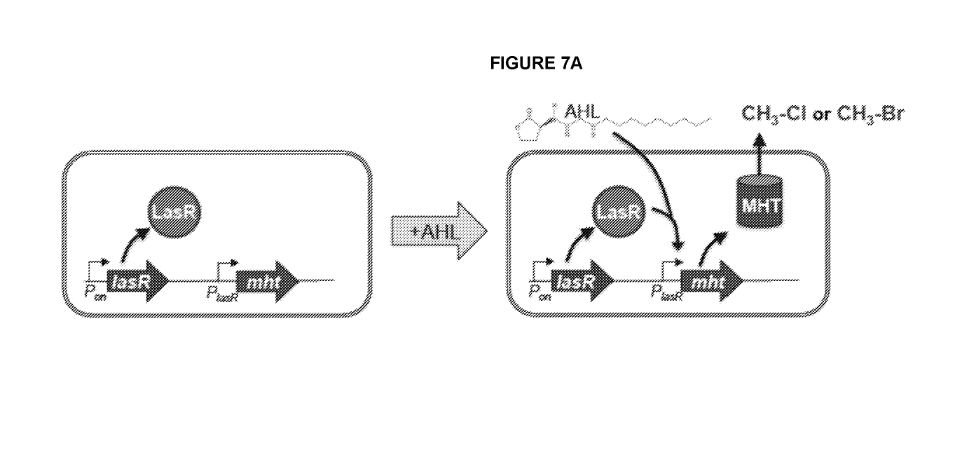

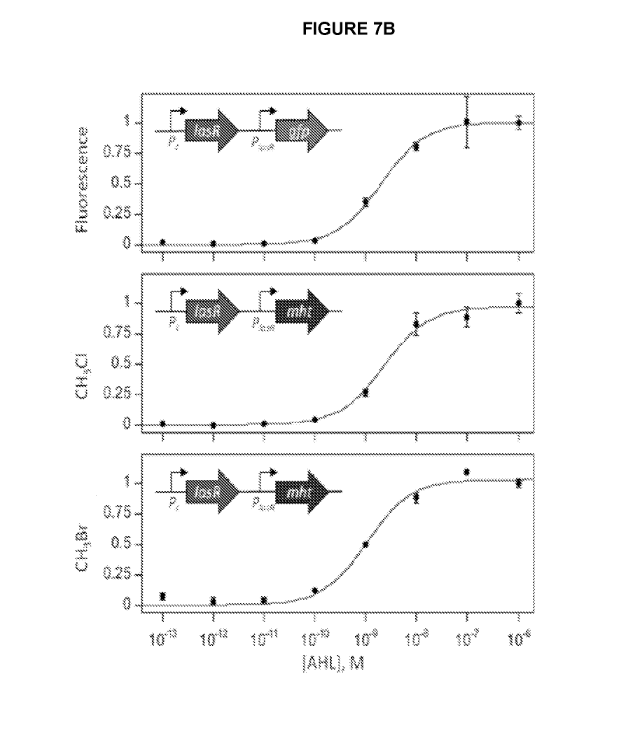

To be useful in soils and other hard-to-image environments, gas-reporting microbes are preferably integrated into the genome so that antibiotics are no longer required to maintain the circuit in cells. Thus, lambda phage recombination was used to incorporate a single copy of the circuit shown in FIG. 7A into the genome of E. coli MG1655. Strains built with this circuit (designated MG1655-19) yielded a strong signal when 100 pM AHL was added to the growth medium (data not shown). However, this signal was approximately half of that observed with cells containing pSH009 plasmid, which encodes the same genetic circuit, presumably because of differences in the copy number of the circuit. Nevertheless, the strong signal and good regulation of MHT in this strain makes MG1655-19 a suitable prototype biosensor for gas-reporting in hard-to-image environments.

### Biochar Assay

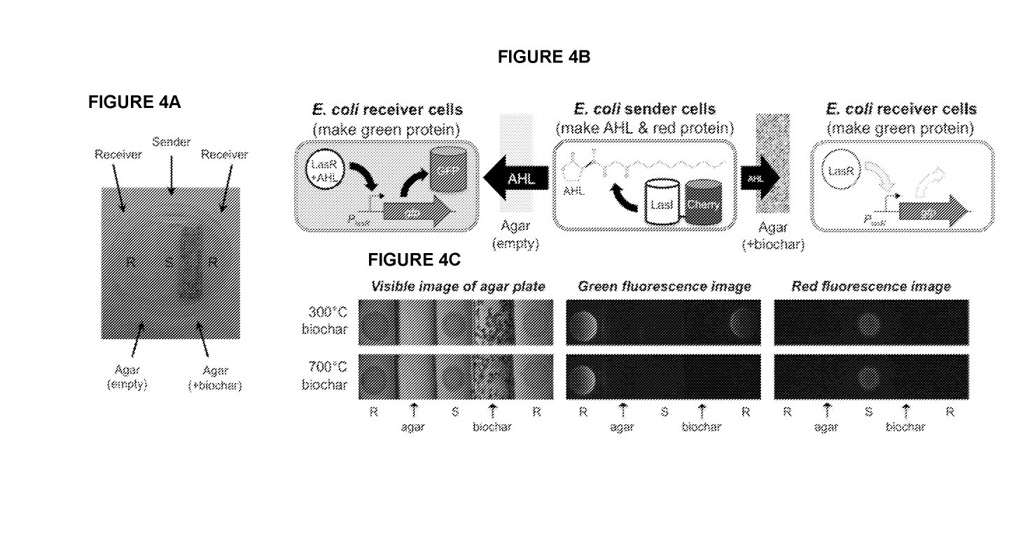

The biosensors can be used in a variety of hard-to-image contexts, for example in evaluating soil, sediment, biochar, partially-opaque media, and the like. Biosensors that generate MHT outputs can help determine why some biochars elicit biological effects upon amendment to soil, whereas others do not. When preparing biochar, many parameters likely influence its ability to sorb biologically-relevant compounds, including the feedstock, temperature, minerals, oxygen, reactor type, and gas flow. Many of these parameters remain poorly constrained, limiting our ability to predict biochar properties upon addition to soil. Furthermore, biochar aging leads to changes in the surface chemistry that controls sorption properties, complicating such predictions. We hypothesize that our microbial biosensors will be useful as a simple, dynamic, non-invasive screen of the sorption of biological signaling molecules to different biochars applied to soils. FIGS. 4A-C shows preliminary work demonstrating 1) the general applicability of biosensors to the problem of biochar sorption and 2) the need for a gas-reporting biosensor.

Using a GFP-reporting sensor we were able to show biochar effects on cell-cell communication in a petri dish. Biochars inhibit cell-cell communication to differing extents in pure culture. FIG. 4A shows an agar plate assay for assessing biochar effects on E. coli sender-receiver communication. Sender and receiver cells were plated on an agar plate such that agar between the each cell type either had low levels of biochar (right) or no biochar (left). In FIG. 4B sender cells spotted between the agar slabs synthesized AHL and receiver cells were spotted outside of both agar slabs and reported on AHL levels by making a GFP reporter. In FIG. 4C images from agar plates containing identical amounts of 300° C. and 700° C. biochars within agar slabs are seen. Bright field images show cell growth, green fluorescence images illustrate how GFP protein expression varies in receiver cells, and red fluorescence images reveal constitutive expression of the protein that synthesizes AHL within the sender cells. Whereas the receiver cells adjacent to the 300° C. biochar exhibited 24.1±2.1% of the GFP fluorescence observed in the receiver cells grown adjacent to agar lacking biochar, the receiver cells grown adjacent to 700° C. biochar displayed only 2.2±1.5% of the GFP fluorescence observed within the receiver cells grown adjacent to empty agar.

Thus, this assay indicates that biochar dose influences cell-cell signaling in petri dishes. However, the behavior of biochars in the environment varies with time and is influenced by the presence of soil minerals, which impact microbial processes. GFP-reporting sensors cannot transmit their signal through a soil matrix, and because of this, cannot be used to provide real-time information on the effects of biochar on soil cell-cell signaling. Gas biosensors, however, can transmit through soils and can be used noninvasively to provide real-time data on microbial behavior. Our data below on matrix assays indicates that the above assay can be easily converted to a MHT-based reporter system that could then be used in soils, sediments and the like providing more relevant data from natural environments.

### Ligand Activated Biosensor

We built a ligand-activated biosensor based on the MHT reporter. Here we chose to use N-acyl homoserine lactone as a ligand because the AHLs are a class of signaling molecules involved in bacterial quorum sensing. Quorum sensing is a method of communication between bacteria that enables the coordination of group-based behavior based on population density. They signal changes in gene expression, such as switching between the flagella gene and the gene for pili for the development of a biofilm. We have used AHL sensitive promoters to control the MHT gene expression as a means of providing biosensors that are sensitive to those species that produce AHLs. In addition, by placing the AHL sensitive MHT in the same bacteria with a constitutive producer of AHL, we can generate a biosensor that is only triggered when the number of bacteria are large enough to trigger MHT production.

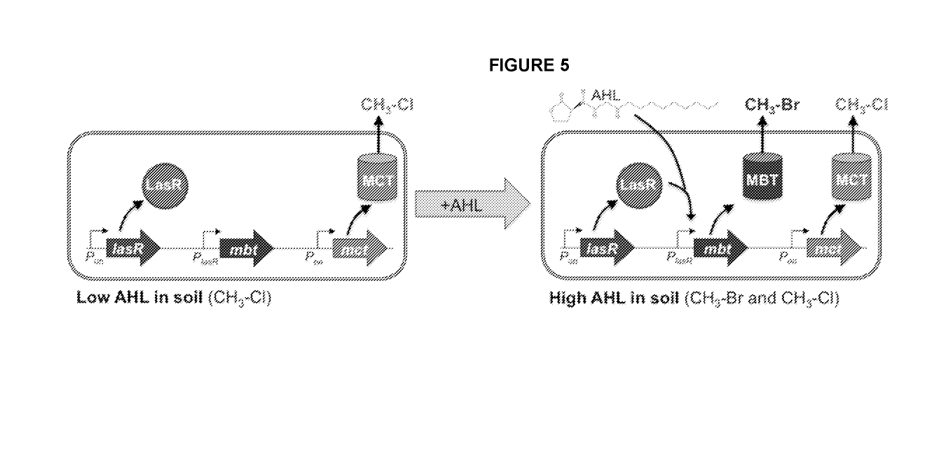

FIG. 5 shows a schematic of one exemplary genetic circuit that programs E. coli to report on AHL within soils. At low cell density (left), cells harboring the genetic circuit synthesize three proteins (LasR, LasI, MCT) and MCT synthesizes CH₃Cl. When cells encounter AHL (right), LasR is activated so that it switches on MBT production and CH₃Br synthesis. CH₃Cl serves as an internal control because the levels detected depend on the number of biosensor cells, the metabolism of those cells, and the fraction of CH₃X that is consumed in soil. The ratio of CH₃Br/CH₃Cl represents an output that is independent of these parameters. CH₃Br and CH₃Cl will be simultaneously measured using GC.

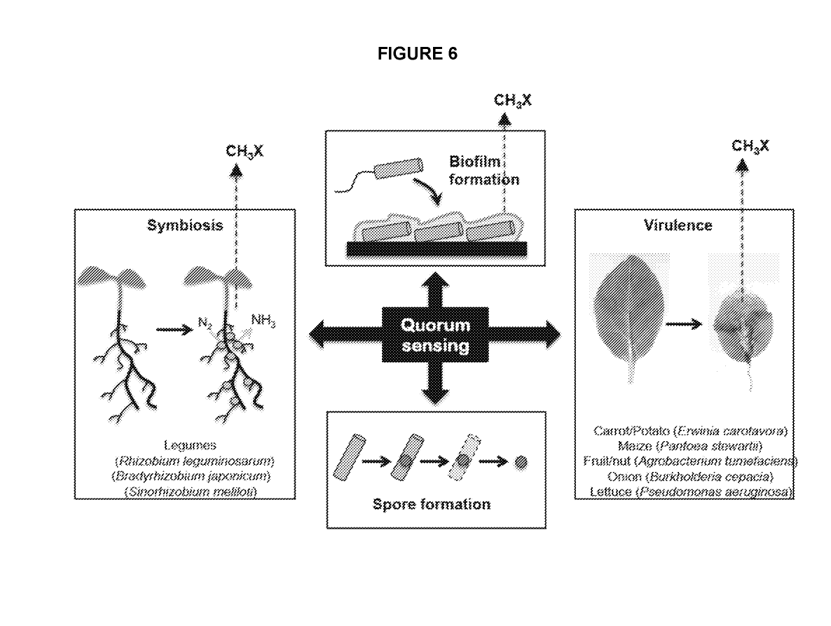

FIG. 6 shows examples of other biological processes that are regulated by quorum sensing. In each of these examples, a gas biosensor could be used to report on the density-dependent phenotypes and the effects of different land use choices on these phenotypes.

FIG. 7A shows a prototype biosensor design that was actually built and tested, wherein the MHT was placed under the control of the AHL responsive promoter lasR from Pseudomonas aeruginosa. Upstream of this was a lasI gene under the control of the constitutive promoter Pcat. As before, the B. maritima MHT gene was used because the protein had high activity with both chloride and bromide ions. We also performed a GFP-based experiment, using the same promoters and vectors as a positive control.

Plasmids pSH001 and pSH009 containing reporter gene gfp and mht respectively were transformed into XL1 E. coli. Fresh LB medium was inoculated with saturated cell culture at OD600=0.005. Cell cultures were induced with AHL at mid-log phase and incubated for 5 hours at 37° C. with 200 mM NaCl or KBr.

GFP measurements were obtained using a Tecan plate reader at excitation at 488 nm and emission at 509 nm. Evolved methyl chloride or methyl bromide gas was measured by an Agilent 7890 GC-MS. All data were normalized to each sample's cell density and scaled to a [0,1] range. Hill function fits were performed on the data (black lines). Error bars were calculated from the standard deviation of three independent experiments.

The results are shown in FIG. 7B, which shows measuring GFP (upper); CH₃Cl (middle) and CH₃Br (lower) at 5 hours with increasing amounts of AHL being added to the cells along with 200 mM Br or Cl substrate as appropriate for use as the MHT substrate. Pₗₐₛ_{_}MHT has a similar AHL dose response curve to Pₗₐₛ_{_}GFP.

Although these experiments are bench-top proof of concept experiments, this result demonstrates that such a biosensor can provide dose-dependent response of chemicals in the environment it resides.

### NADPH Biosensor

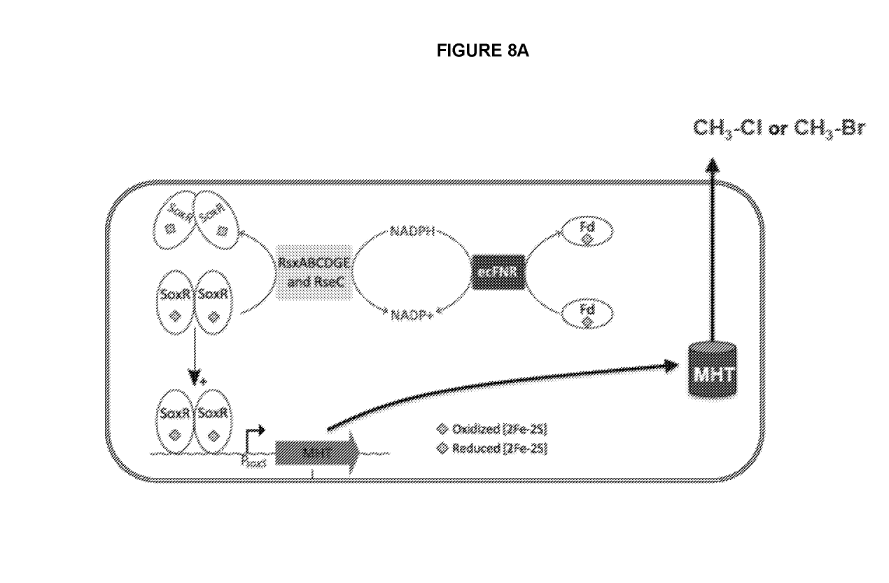

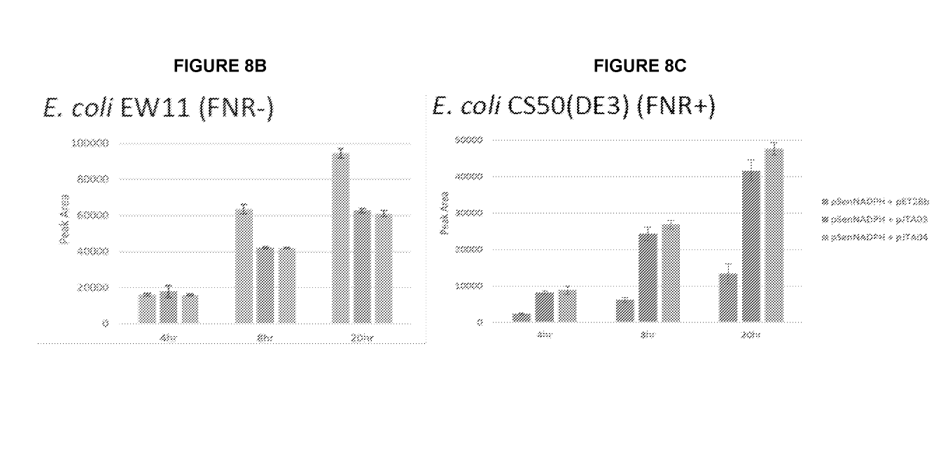

FIG. 8A shows the design for an E. coli biosensor for cellular NADPH levels. To translate NADPH/NADP⁺ level to a measurable output signal, we fused P_{soxS} (PMID: 24283989) to Batis maritima MI-1T (BmMHT). The activation P_{soxS} is governed by the oxidative status of a [2Fe-2S] cluster-containing transcriptional regulator, SoxR. Indigenous NADPH-dependent reductases (e.g., RsxABCDGE and RseC) keep SoxR in its reduced state, which does not activate PsoxS. When cellular NADPH decreases (e.g., NADP⁺ increases), SoxR can no longer be maintained in reduced state, and the oxidized SoxR activates PsoxS. The plasmid, pSenNADPH, encodes a P_{soxS}-BmMHT fusion protein. To decrease cellular NAPDH concentration, we overexpressed ferredoxins in cells by transforming plasmid pJTA03 encoding soFd (Plant type [2Fe-2S] Ferredoxin from Spinacia oleracea) or pJTA03 encoding crFd(Plant type [2Fe-2S] Ferredoxin from Chlamydomonas reinhardtii). Ferredoxins serves as NADPH sinks in the cell because E. coli ferredoxin-NADPH reductase (ecFNR) reduces ferredoxins and oxidize NADPH to NADP⁺. This reaction counteracts with SoxR reduction mechanism and shifts SoxR to the oxidized state. Therefore, by overexpressing soFd or crFd, we should be able to observe a production of methyl halides.

FIG. 8B and FIG. 8C show a proof-of-concept results for the E. coli biosensor for detecting cellular NADPH levels. In ecFNR expressed E. coli CS50 (DE3) strain (FIG. 8C), the methyl bromide productions were increased when soFd or crFD was co-transformed with pSenNADPH. In ecFNR deleted EW11 strain (FIG. 8B), the same increment was not observed. These results show that pSenNADPH can report on cellular NADPH availability, which could developed into useful tools to monitor cellular metabolic states, which can be beneficial to metabolic engineering.

### Quorum Biosensor

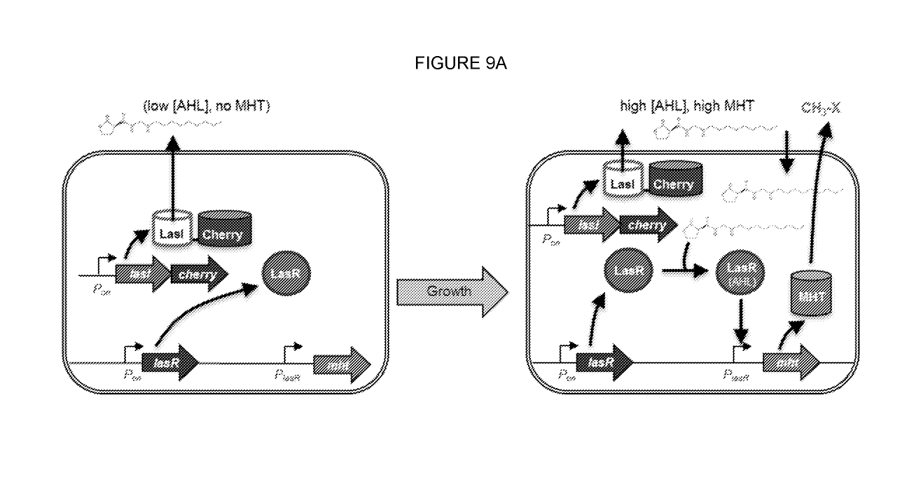

FIG. 9A shows the design for an E. coli biosensor for quorum sensing using a constitutive lasI from Pseudomonas aeruginosa to produce the AHL needed to turn on the MHT gene. Because a constitutive promoter is used to regulate lasI transcription, cells always produce N-3-oxo-dodecanoyl-L-homoserine (3oxoC₁₂HSL). Therefore, the concentration of 3oxoC₁₂HSL increases with the size of the population of bacteria in a culture, and the circuit is designed to turn “on” the methyl halide gas output when the population reaches a threshold cell density.

To couple this AHL input to a CH₃X output, the transcriptional regulator LasR that has evolved to respond to LasI was used to control the transcription of an MHT. We chose this design because it mimics a quorum sensing system found in many soil bacteria, which use accumulation of AHL to change transcription of multiple genes at specific cell population densities to coordinate gene expression within a population. We will also continuously express a red fluorescent protein (Cherry) as a cell growth control, although cell density can be determined by culture absorbance at 600 nm.

We built this genetic circuit using Batis maritima MHT, because it has the highest known methyl halide production rate in E. coli. The genes that make up each circuit were constructed by PCR amplifying the LasI, LasR, and Cherry genes from plasmids previously available in our lab and by commercially synthesizing each MHT. Each gene was built as a fusion to its promoter and ribosomal binding site, and then cloned into plasmids using e.g., Golden gate assembly. E. coli were transformed with each vector to create a microbe whose CH₃X synthesis depends on cell density.

We grew our E. coli sensor within gas tight culture tubes containing halide salts and LB medium to show that CH₃X production can be used as a quorum-sensing reporter. We did this by measuring: (i) CH₃X production, and (ii) Cherry as a proxy for cell density.

Cultures were started at a low cell density where MHT was not expressed, and each parameter measured by removing aliquots at different times for analysis. Cell density was alternately quantified by OD600 or by measuring Cherry fluorescence in whole cells (λₑₘ=610 nm) using a Tecan M1000 plate reader.

CH₃X levels in the headspace of cultures were measured using a gas chromatograph (GC) system as described. Because Cherry is continuously made within cells, CH₃X/Cherry or CH₃X/OD ratios will report on changes in the per cell levels of CH₃X.

The experimental results are shown in FIG. 9B. As can be seen, cell density normalized CH₃Br increased with the density of the cell population once quorum levels were reached.

### Matrix Assays

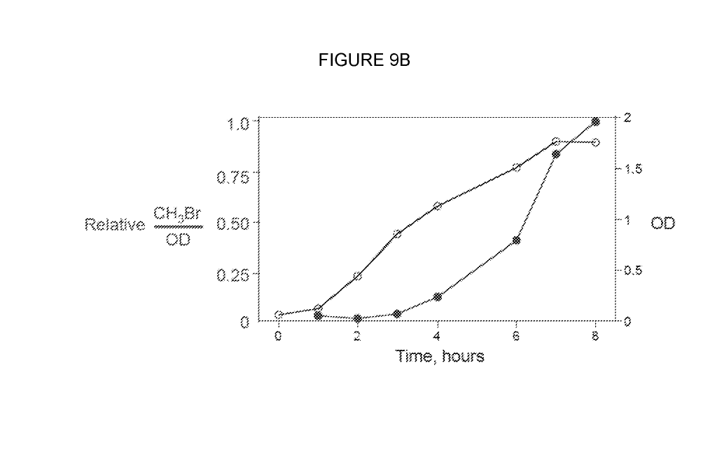

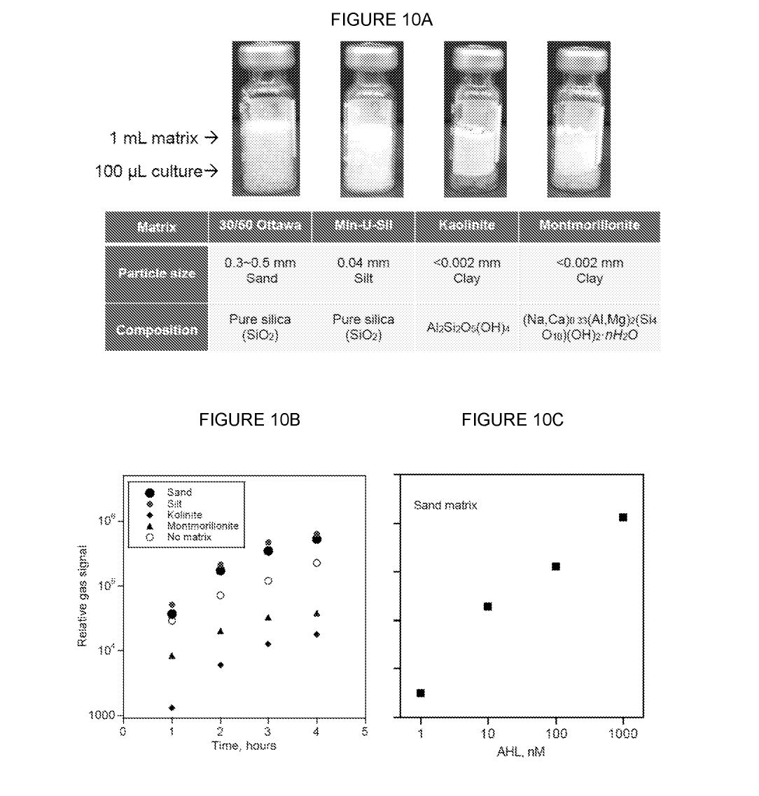

In order to be useful for testing microbes in situ environments, the methyl halide gas reporters must still work in soils and other matrices. To examine the matrix effect on CH₃X production by bioreporters, we incubated the integrated LasR-regulated MHT bioreporter, MG1655-19 E. coli strain with matrices supplemented with AHL that represent four most common types of particle sizes in soil, including sand, silt, and clay. See FIG. 10A. Four matrices with different particle sizes and chemical compositions were tested. Four identical samples were prepared for each matrix. At each time point, one sample from each group was analyzed for headspace CH₃Br concentration.

FIG. 10B is a time response for LasR-regulated MHT reporters within a porous matrix to which AHL was added. It shows that the intensity of the gas signals from the sand and silt matrices are similar to the group without matrix, and lower output signals in two clay matrices, which might due to sub-optimal growth condition resulted from less available water to bacteria.

To further investigate if LasR-regulated MHT bioreporter retains its function of detecting AHL in a soil matrix, we conducted a preliminary AHL experiment in a sandy matrix that examined the dose response. We found that the CH₃Br output signal increased as AHL concentration increasing from 1 nM to 1 mM (FIG. 10C).

These two pilot experiments demonstrate the possibility of using gas bioreporters to directly report in a solid, porous matrix. Additional work is required to understand the extent to which methyl halides are sorbed to these matrices on the time course of biosensing experiments before the biosensors can be used for real applications, but these proof of concept experiments predict a strong likelihood of success in using the gas reporting biosensors in natural environments.

### Toxicity Biosensor

Biosensors have been developed that provide microbial perspectives on the levels of chemicals in environmental samples, which are often distinct from the total concentration quantified using analytical chemistry measurements. As listed in Table 2, diverse biosensors have been constructed to detect heavy metals, organic pollutants, and nutrients for environmental diagnostic applications. However, most of these biosensors remain in the proof-of-concept stage and are not suitable for dynamic reporting in hard-to-image samples. This gap can be solved by coupling MHT to existing sensing mechanisms to construct biosensors the reports on chemical concentrations in hard-to-image samples.

For toxicity monitoring, as an example, a selected promoter from a bacterial toxic response network can be operably fused to an MHT reporter gene and integrated back into the host cell. The additional promoter-reporter fusion will therefore behave, ideally, as an integral part of the correct cellular toxicity response network, and reporter induction can be seen as representative of the targeted response.

### Metal Biosensor

There are (at least) two metal sensing repressors known to respond to cadmium: ArsR and CzrA from Bacillus subtilus. However, their specificity for cadmium is not unique. ArsR also detects arsenic and CzrA also detects zinc and copper. The table below shows the metals which release both ArsR and CzrA from their DNA binding sites:

| Metal Sensor | Metals Sensed | | | |
|---|---|---|---|---|
| ArsR | As(III) | Ag(I) | Cu | Cd |
| CzrA | Zn | Co | Ni | Cd |

By positioning the operator binding sites for these two metal sensing repressors next to each other in a promoter region, the gene regulated by that promoter will be transcribed only when a metal that binds to both sensors is present—in this case Cadmium. Thus, by combining both promoters with an MHT gene, we can develop cells that become sensitive cadmium sensors. Preferably, the construct is integrated into the genome of the host cell so that antibiotic selection is no longer needed. The cells can thus be seeded into those environments where cadmium detection is desired. For arsenic detection, ArsR can be used alone.

The same principles can be used for other metal responsive promoters, e.g., nrsB and arsB from Cyanobacterium synechocystis PCC 6803; merR for mercury; cadC for cadmium, pbrR for lead, znt for cadmium, lead and zinc, cnr for cobalt and nickel, Ace 1, copA or cup1 for copper, and the like.

### Radiation Biosensor

Certain promoters are known to respond to radiation, and biosensors have been built based on these promoters. For example, recA, grpE and katG promoters respond to radiation. Workers have already developed an E. coli DPD2794 biosensor using recA::luxCDABE, and were able to detect as little as 1.5 Gy gamma irradiation, while the maximum response was obtained at 200 Gy. Replacing of the lux reporter with an MHT reporter would provide a gas reporter based biosensor for radiation.

### Steroid Biosensor

A variety of promoters are known to respond to estrogenic or androgenic compounds, which present serious environmental concerns due to their profound effects on mammalian biology. For example, there are several hER and hAR receptors that are steroid responsive, and these can have been successfully used to make biosensors in yeast A. adeninivorans.

For example, the following biosensor is already commercialized, and could be easily modified to use gas reporters. The microbial component of the biosensor consists of transgenic A. adeninivorans yeast cells. Integrated in the genome these cells carry a receptor gene cassette with e.g., a TEF1-promotor—hERα gene—PHO5-terminator and a reporter gene cassette with GAA-ER6-promotor—reporter gene—PHO5-terminator. The TEF1-promotor provides constitutive expression of the receptor gene, which leads to the synthesis of the recombinant estrogen receptor α (hERα). When incubated with estrogenic substances, the hERα is expressed and forms dimers which act as transcription factors that bind to the ERE region of the GAA-ERE-promoters. Consequently the expression of the reporter gene (which is phyK in the commercial assay, but could be WIT as described herein) is activated leading to synthesis of the reporter protein (Phytase or MHT) whose activity can be measured subsequently.

### CO₂ Biosensor

A CO₂ biosensor was developed using the CO₂ responsive promoter sequence of the chloroplastic carbonic anhydrase gene in P. tricornutum (Pptcal). A Pptcal with a deleted initiator region was ligated with the minimal region of the PCMV followed by uidA, which encodes GUS, and was introduced into P. tricornutum. GUS expression in the resulting transformants was clearly regulated by CO₂, that is, GUS expression was stimulated in air (about 0.04% CO₂) about 10-fold less than that in cells grown in 5% CO₂.

Replacement of GUS with MHT will provide a sensitive gas-based biosensor. While the above biosensor was developed for use in the marine diatom, similar principles can be applied to other species. In particular, a species suitable for use in coal-mines could provide a canary biosensor for the early detection of toxic gases, such as CO₂, and methane responsive promoter could detect methane.

### Toluene Biosensor

A toluene biosensor using a light-based reporter has already been developed, and can be easily modified to produce gas reporters. The transcriptional activator xylR from the TOL plasmid of Pseudomonas putida mt-2 was used. The XylR protein binds a subset of toluene-like compounds and activates transcription at its promoter, Pᵤ. A reporter plasmid was constructed by placing firefly luciferase under the control of XylR and Pᵤ. When E. coli cells were transformed with this plasmid vector, luminescence from the cells was induced in the presence of benzene, toluene, xylenes, and similar molecules. Accurate concentration dependencies of luminescence were obtained and exhibited K_{1/2} values ranging from 39.0±3.8 μM for 3-xylene to 2,690±160 μM for 3-methylbenzylalcohol (means±standard deviations). The luminescence response was specific for only toluene-like molecules that bind to and activate XylR.

These biosensor cells were field tested on deep aquifer water, for which contaminant levels were known, and were able to accurately detect toluene derivative contamination in water. The biosensor cells were also shown to detect BETX (benzene, toluene, and xylene) contamination in soil samples. These results demonstrate the capability of such a bacterial biosensor to accurately measure environmental contaminants and suggest a potential for its inexpensive application in field-ready assays. Changing this light based reporter to a gas based detection system would allow easier field implementation of such as biosensor.

### Inorganic Biosensors

A variety of promoters are known to respond to inorganic molecules, and can be used as described above to create sensors for detecting the presence of inorganic compounds. For example, nar and nblA have both been used with fluorescent reporters to detect nitrates. These can easily be combined with the MHT genes herein, and used in gas-based biosensors for nitrate.

### Dual Biosensors

A two-level or two-chemical biosensor can be developed by equipping microbes with two MHT genes having different substrate specificities, under promoters responsive to different chemicals or different levels of the same chemical. For example, methane monooxygenase (MMO) are found in methanotrophs and catalyze the oxidation of methane to methanol, allowing these bacteria to use methane as a sole carbon and energy source. There are two distinct types of MMO enzymes: a cytoplasmic soluble enzyme (sMMO) and a membrane-bound particulate enzyme (pMMO) and both are regulated by copper levels. In cells that synthesize both types of enzyme, sMMO is expressed at low copper-biomass ratios, while pMMO is expressed at high copper-biomass ratios. These two promoters could thus be used to establish a dual level copper biosensor, wherein low levels of copper result in the production of e.g., CH₃Cl and high levels result in the production of CH₃Br. This same principle can be applied to any of the biosensors herein to detect either different chemicals or different levels of the same chemical. Likewise, the principle can be extended to include three MHTs with different substrate specificity.

### Additional Biosensors

A variety of stress responsive networks that have been exploited to develop biosensors are shown in TABLE 2. Any of these systems can be combined with the gas reporter genes described in the above examples, thus producing biosensors that are better suited for field work and other environments where light based reporters are unsuitable.

TABLE 2
Proteins used for bacterial bioreporter construction (3)

| | | Promoter- | | Detection | |
|---|---|---|---|---|---|
| Sensor protein | Host chassis | reporter fusion | Chemical targets | sensitivity | |
| XylR of | Escherichia | Pu*-lucFF | Benzene, toluene | 40 | μM |
| Pseudomonas | coli | | and Xylene | | |
| putida | | | | | |
| DmpR of | P. putida | Po^{‡}-luxAB | Phenol | 3 | μM |
| P. putida | | | | | |
| TbuT of | E. coli | tbuAlp-luxAB | Benzene, toluene | 0.24 | μM |
| Ralstonia | | | and Xylene | | |
| pickettii | | | | | |
| HbpR of | E. coli | hbpCp-luxAB | Hydroxylated | 0.4 | μM |
| Pseudomonas | | | biphenyls | | |
| nitroreducens | | | | | |
| PhnR of | B. sartisoli | phnSp-luxAB | Naphthalene and | 0.17 | μM |
| Burkholderia | | | phenanthrene | | |
| sartisoli | | | | | |
| IbpR of | E. coli | ibpAp- | Various aromatics | 1 | μM |
| P. putida | | luxCDABE | | | |
| NahR of | P. putida | nahGp-luxAB | Naphthalene and | 10 | nM |
| P. putida | | | salicylate | | |
| AlkS of | E. coli | alkBp-luxAB | C₆-C₁₀ alkanes | 10 | nM |
| Pseudomonas | | | | | |
| oleovorans | | | | | |
| TodST of | P. putida | todXp- | Toluene, benzene, | 0.3 | μM |
| P. putida | str. F1 | luxCDABE | phenol, p-xylene, | | |
| | | | m-xylene and | | |
| | | | trichloroethene | | |
| SepR of | P. putida | sepAp- | Solvents | ~0.5 | mM |
| P. putida | str. F1 | luxCDABE | | | |
| FruR of Erwinia | E. herbicola | JruBp- | Fructose and | ~2 | μM |
| herbicola | | gfp[AAV]^{§} | sucrose | | |
| AraC of E. coli | E. coli | pBAD-gfpuv^{\|\|} | L-Arabinose | 0.5 | μM |
| ArsR of E. coli | E. coli | arsRp-luxAB | Arsenite and | 5 | nM |
| | | | antimonite | | |
| MerR of E. coli | E. coli | merTp- | Hg²⁺ | 1 | nM |
| | | luxCDABE | | | |
| CadC of | Bacillus | cadCp-lucFF | Cd^{2+,} Pb, Sn and | 3 | nM |
| Staphylococcus | subtilis | | Zn | | |
| aureus | | | | | |
| ZntR of E. coli | E. coli | zntAp- | Zn, Pb and Cd | 5 μM, 0.7 μM and | |
| | | luxCDABE | | respectively | |
| TetR of E. coli | E. coli | tetAp- | Tetracyclines | 45 | nM |
| | | luxCDABE | | | |
| MphR of E. coli | E. coli | mphAp-lacZ | Macrolides (such | ~10 | μM |
| | | | as erythromycin) | | |
| SOS response | B. subtilis | yorBp-lucFF | Various antibiotics | 60 | nM |
| proteins of | | | (for example, | | |
| B. subtilis | | | ciprofloxacin) | | |
| SpoIIID and σ^{E} | B. subtilis | yheI-lucFF | Various antibiotics | 0.1 | μM |
| of B. subtilis | | | (e.g., linezolid) | | |
| NisRK of | L. lactis | nisAp-gfpuv^{\|\|} | Nisin | 10 ng l¹ (3 pM) in | |
| Lactococcus | | | | culture supernatant | |
| lactis | | | | and 0.2 μg l⁻¹ | |
| | | | | (60 pM) in milk | |
| LuxR of | E. coli | luxIp-gfp[ASV]^{§} | N-Acyl homoserine | 1-10 | nM |
| Aliivibrio | | | lactones | | |
| fischeri | | | | | |

| | | | | |
|---|---|---|---|---|
| Ada of E. coli | E. coli | alkAp- | DNA-alkylating | 70 nM N-methyl- |
| | | luxCDABE | agents | N′-nitro-N- |
| | | | | nitrosoguanidine, |
| | | | | for example |
| DnaK and σ32 | E. coli | dnaKp- | An increase in the | 0.25M methanol, |
| of E. coli | | luxCDABE | level of intracellular | for example |
| | | | misfolded proteins | |
| Crp-cAMP | E. coli | grpEP- | An increase in the | 0.14 nM |
| transcriptional | | luxCDABE | level of intracellular | pentachlorophenol, |
| dual regulator | | | misfolded proteins | for example |
| of E. coli | | | | |
| OxyR of E. coli | E. coli | katGp- | Intracellular | 3 μM H₂O₂, |
| | | luxCDABE | production of | for example |
| | | | oxygen radicals | |
| SoxRS of E. coli | E. coli | micFp- | Intracellular | Detection |
| | | luxCDABE | production of | sensitivity not |
| | | | oxygen radicals | indicated |
| RecA-LexA of | E. coli | cdap-gfp | Single-stranded | 5 nM N-methyl- |
| E. coli | | | DNA that arises as | N′-nitro-N- |
| | | | a consequence of | nitrosoguanidine, |
| | | | inhibition of DNA | for example |
| | | | replication | |
| | | sfiA-lacZ | Single-stranded | 4 nM mitomycin C |
| | | | DNA that arises as | |
| | | | a consequence of | |
| | | | inhibition of DNA | |
| | | | replication | |
| | | reAp- | Single-stranded | 0.2 nM mitomycin C |
| | | luxCDABE | DNA that arises as | |
| | | | a consequence of | |
| | | | inhibition of DNA | |
| | | | replication | |
| RecA-LexA of | S. | recNp- | Single-stranded | 46 nM mitomycin C |
| Salmonella | Typhimurium | luxCDABE | DNA that arises as | |
| enterica subsp. | | | a consequence of | |
| enterica | | | inhibition of DNA | |
| serovar | | | replication | |
| Typhimurium | | umuDp-lacZ | Single-stranded | 10 nM mitomycin C |
| | | | DNA that arises as | |
| | | | a consequence of | |
| | | | inhibition of DNA | |
| | | | replication | |

AraC, arabinose operon regulatory protein; cdap, promoter of the colicin D gene; cAMP, cyclic AMP; Crp, cAMP regulatory protein; katGp, promoter of the catalase-peroxidase gene; lacZ, β-galactosidase gene; lucFF, firefly luciferase gene; lux, bacterial luciferase biosynthesis gene; Rec, recombination and repair; sfiA, SOS cell division inhibitor gene [also known as sulA); tbuA1p, promoter of the toluene monooxygenase α-subunit gene.
*AXylR-responsive promoter of P. putida.
^{‡}DmpR-responsive promoter of P. putida.
^{‡}Unstable variants of GFP.
^{||}A GFP variant that is optimized for maximal fluorescence when excited by ultraviolet light.

The following references are incorporated by reference in their entirety.
1. Khalil A S, Collins J J (2010) Synthetic biology: applications come of age. Nat Rev Genet 11:367-379.
2. Nandagopal N, Elowitz M B (2011) Synthetic Biology: Integrated Gene Circuits. Science 333:1244-1248.
3. van der Meer J R, Belkin S (2010) Where microbiology meets microengineering: design and applications of reporter bacteria. Nat Rev Microbiol 8:511-522.
4. Herron P M, Gage D J, Cardon Z G (2010) Micro-scale water potential gradients visualized in soil around plant root tips using microbiosensors. Plant Cell Environ 33:199-210.
5. Gage D J, Herron P M, Arango Pinedo C, Cardon Z G (2008) Live reports from the soil grain—the promise and challenge of microbiosensors. Funct Ecology 22:983-989.
6. Diplock E E, Alhadrami H A, Paton G I (2009) in eds Diplock E E, Alhadrami H A, Paton G I (Springer Berlin Heidelberg, Berlin, Heidelberg), pp 189-209.
7. Giepmans B N G (2006) The Fluorescent Toolbox for Assessing Protein Location and Function. Science 312:217-224.
8. DeAngelis K M, Firestone M K, Lindow S E (2007) Sensitive Whole-Cell Biosensor Suitable for Detecting a Variety of N-Acyl Homoserine Lactones in Intact Rhizosphere Microbial Communities. Appl Environ Microb 73:3724-3727.
9. DeAngelis K M, Ji P, Firestone M K, Lindow S E (2005) Two Novel Bacterial Biosensors for Detection of Nitrate Availability in the Rhizosphere. Appl Environ Microb 71:8537-8547.
10. Schmidberger J W, James A B, Edwards R, Naismith J H, O'Hagan D (2010) Halomethane Biosynthesis: Structure of a SAM-Dependent Halide Methyltransferase from Arabidopsis thaliana. Angew Chem Int Ed 49:3646-3648.
11. Paul C, Pohnert G (2011) Production and role of volatile halogenated compounds from marine algae. Nat Prod Rep 28:186.
12. NiX, Hager L P (2013) Expression of Batis maritima methyl chloride transferase in Escherichia coli. P Natl Acad Sci Usa 96:3611-3615.
*13. Bayer T S et al. (2009) Synthesis of Methyl Halides from Biomass Using Engineered Microbes. J Am Chem Soc 131:6508-6515.
14. Miller M B, Bassler B L (2001) Quorum sensing in bacteria. Annual Reviews in Microbiology 55:165-199.
15. Wagner V E, et al., (2003) Microarray Analysis of Pseudomonas aeruginosa Quorum-Sensing Regulons: Effects of Growth Phase and Environment. Journal of Bacteriology 185:2080-2095.
16. DeLisa M P, et al., (2001) DNA Microarray-Based Identification of Genes Controlled by Autoinducer 2-Stimulated Quorum Sensing in Escherichia coli. Journal of Bacteriology 183:5239-5247.
17. Gonzalez J E, Marketon M M (2003) Quorum Sensing in Nitrogen-Fixing Rhizobia. Microbiology and Molecular Biology Reviews 67:574-592.
18. Masiello C A Ye, C, Gao, X, Liu, S, Cheng, S, Bennett, M R, Rudgers, J A, Zygourakis, K, Wagner, D S, and Silberg, J J (2013) Pyrolysis temperature determines biochar effects on microbial communication. Environmental Science & Technology, 47:11496-11503.
US20110151534.

1. A method, comprising:
a) growing a microorganism comprising an exogenous gene encoding a methyl halide transferase (MHT) in a solid matrix or a partially opaque medium, wherein said gene is under the direct or indirect control of a promoter-of-interest;
b) adding a substrate for said WIT to said solid matrix or a partially opaque medium, wherein said WIT converts said substrate to a gas;
c) capturing said gas released by said microorganism; and
d) measuring an amount of said captured gas, said amount being proportional to an activity of said promoter-of-interest.

2. The method of claim 1, wherein said exogenous gene is genomically integrated into said microorganism.

3. The method of claim 1, wherein said exogenous gene encodes a protein having SEQ ID NO. 1.

4. The method of claim 1, wherein said substrate is a chlorine or bromine or iodine ion.

5. The method of claim 1, wherein said exogenous gene is from Batis maritima.

6. The method of claim 1, wherein said exogenous gene encodes a WIT having a sequence selected from:
SEQ ID NO: 1
MSTVANIAPV FTGDCKTIPT PEECATFLYK VVNSGGWEKC
WVEEVIPWDL GVPTPLVLHL VKNNALPNGK GLVPGCGGGY
DVVAMANPER FMVGLDISEN ALKKARETFS TMPNSSCFSF
VKEDVFTWRP EQPFDFIFDY VFFCAIDPKM RPAWGKAMYE
LLKPDGELIT LMYPITNHEG GPPFSVSESE YEKVLVPLGF
KQLSLEDYSD LAVEPRKGKE KLARWKKMNN
SEQ ID NO: 2
MASAIVDVAG GGRQQALDGS NPAVARLRQL IGGGQESSDG
WSRCWEEGVT PWDLGQPTPA VVELVHSGTL PAGDATTVLV
PGCGAGYDVV ALSGPGRFVV GLDICDTAIQ KAKQLSAAAA
AAADGGDGSS SFFAFVADDF FTWEPPEPFH LIFDYTFFCA
LHPSMRPAWA KRMADLLRPD GELITLMYLA EGQEAGPPFN
TTVLDYKEVL NPLGLVITSI EDNEVAVEPR KGMEKIARWK
RMTKSD
SEQ ID NO: 3
MAGPTTEFWQ ERFEKKETGW DRGSPSPQLL AWLASGALRP
CRIAVPGCGS GWEVAELAQR GFDVVGLDYT AAATTRTRAL
CDARGLKAEV LQADVLSYQP EKKFAAIYEQ TCLCAIHPDH
WIDYARQLHQ WLEPQGSLWV LFMQMIRPAA TEEGLIQGPP
YHCDINAMRA LFPQKDWVWP KPPYARVSHP NLSHELALQL
VRR
SEQ ID NO: 4
MENVNQAQFW QQRYEQDSIG WDMGQVSPPL KAYIDQLPEA
AKNQAVLVPG AGNAYEVGYL HEQGFTNVTL VDFAPAPIAA
FAERYPNFPA KHLICADFFE LSPEQYQFDW VLEQTFFCAI
NPSRRDEYVQ QMASLVKPNG KLIGLLFDKD FGRDEPPFGG
TKDEYQQRFA THFDIDIMEP SYNSHPARQG SELFIEMHVK
D
SEQ ID NO: 5:
MAEVQQNSGN SNGENIIPPE DVAKFLPKTV DEGGWEKCWE
DGVTPWDQGR ATPLVVHLVE SSSLPLGRGL VPGCGGGHDV
VAMASPERYV VGLDISESAL EKAAETYGSS PKAKYFTFVK
EDFFTWRPNE LFDLIFDYVV FCAIEPETRP AWAKAMYELL
KPDGELITLM YPITDHDGGP PYKVAVSTYE DVLVPVGFKA
VSIEENPYSI ATRKGKEKLA RWKKIN
SEQ ID NO: 6
MNLSADAWDE RYTNNDIAWD LGEVSSPLKA YFDQLENKEI
KILIPGGGNS HEAAYLFENG FKNIWVVDLS ETAIGNIQKR
IPEFPPSQLI QGDFFNMDDV FDLIIEQTFF CAINPNLRAD
YTTKMHHLLK SKGKLVGVLF NVPLNTNKPP FGGDKSEYLE
YFKPFFIIKK MEACYNSFGN RKGRELFVIL RSK
SEQ ID NO: 7
MSDPTQPAVP DFETRDPNSP AFWDERFERR FTPWDQAGVP
AAFQSFAARH SGAAVLIPGC GSAYEAVWLA GQGNPVRAID
FSPAAVAAAH EQLGAQHAQL VEQADFFTYE PPFTPAWIYE
RAFLCALPLA RRADYAHRMA DLLPGGALLA GFFFLGATPK
GPPFGIERAE LDALLTPYFD LIEDEAVHDS IAVFAGRERW
LTWRRRA
and
SEQ ID NO: 8
MTDQSTLTAA QQSVHNTLAK YPGEKYVDGW AEIWNANPSP
PWDKGAPNPA LEDTLMQRRG TIGNALATDA EGNRYRKKAL
VPGCGRGVDV LLLASFGYDA YGLEYSGAAV QACRQEEKES
TTSAKYPVRD EEGDFFKDDW LEELGLGLNC FDLIYDYTFF
CALSPSMRPD WALRHTQLLA PSPHGNLICL EYPRHKDPSL
PGPPFGLSSE AYMEHLSHPG EQVSYDAQGR CRGDPLREPS
DRGLERVAYW QPARTHEVGK DANGEVQDRV SIWRRR.

7. A method of detecting the activity of a promoter-of-interest, comprising adding a microorganism comprising an exogenous gene encoding a methyl halide transferase (MHT) to a sample, wherein said MHT gene is under the direct or indirect control by a promoter-of-interest, adding a halide salt to said sample, incubating said sample until said halide salt is converted to a gaseous methyl halide, and detecting an amount of said methyl halide gas being emitted from said sample, wherein said amount directly correlates to an activity level of said promoter-of-interest.

8. The method of claim 7, wherein said exogenous gene encoding said MHT is integrated into the genome.

9. The method of claim 7, wherein said promoter-of-interest is a metal-sensing promoter.

10. The method of claim 7, wherein said promoter-of-interest is a stress-sensing promoter.

11. The method of claim 7, wherein said promoter-of-interest is a redox-sensing promoter.

12. The method of claim 7, wherein said promoter-of-interest is an estrogen- or androgen-responsive promoter and wherein said microorganism also comprises an exogenous gene for an estrogen receptor or an androgen receptor, respectively.

13. The method of claim 7, wherein said promoter-of-interest is an aromatic hydrocarbon responsive promoter.

14. The method of claim 13, wherein said promoter-of-interest is a benzene, toluene, and xylene (BETX) responsive promoter.

15. The method of claim 1, wherein said promoter is activated by a ligand.

16. The method of claim 1, wherein said exogenous gene is activated by a transcriptional activator.

17. The method of claim 1, wherein said transcriptional activator is lasR, xylR, SoxR, or AHL

18. The method of claim 7 wherein said exogenous gene is activated by a transcriptional activator.

19. The method of claim 7, wherein said transcriptional activator is lasR, xylR, SoxR, or AHL
